(19) United States
(12) Patent Application Publication
(10) Pub. No.: US 2016/0367515 A1
(43) Pub. Date: Dec. 22, 2016

(54) Improved delivery of poorly water soluble drugs with aphafetoprotein stabilized with metal ions

(71) Applicant: Oy Safemed Ltd., Aura (FI)

(72) Inventors: Elena Dudich, Lyubuchany (RU); Igor Vyacheslavovich Dudich, Lyubuchany (RU); Lidia Nikolaevna Semenkova, Lyubuchany (RU); Eduard Tatulov, Moscow (RU); Timo Korpela, Turku (FI)

(73) Assignee: Oy Safemed Ltd., Aura (FI)

(21) Appl. No.: 15/105,931

(22) PCT Filed: Nov. 18, 2014
(86) PCT No.: PCT/FI2014/000034

§ 371 Date: Jun. 17, 2016

(30) Foreign Application Priority Data
Nov. 19, 2013 (FI) 20130341

(51) Int. Cl. A61K 31/337; A61K 31/12; A61K 33/30

(52) U.S. Cl. CPC A61K 31/337; A61K 33/30; A61K 31/12

(57) ABSTRACT

The invention discloses a novel significantly improved carrier protein for pharmaceutical applications: metal ion-stabilized alfa-fetoprotein, AFP. The stabilized AFP serves as a novel universal drug delivery vehicle in formulating drugs to be targeted to cancer cells. Various transition metal ions form non-covalent adducts with AFP and stabilize it against physical and chemical factors in physiological fluids while not affecting its ability to target to AFP receptor-expressing cells.

### FIELD OF THE INVENTION

This invention relates to pharmaceutical compositions comprising a drug and a targeting carrier protein with other formulation agents. Pharmaceutical compositions of the present invention are intended for use in targeting therapy, especially in oncology. Whereas alpha-fetoprotein (AFP) can act as the targeting drug carrier protein, according to the present invention, its properties can be significantly improved by allowing it to react with certain transition metal ions. Synergistic biological effects can exist between AFP, metal ions, and drugs.

### BACKGROUND OF THE INVENTION

Alpha-fetoprotein (AFP) is the major component of embryonic blood serum protein of mammals. AFP is synthesized by embryonic liver and yolk sac during the perinatal development. Immediately after the birth, the level of AFP in the serum sharply decreases and its expression becomes undetectable. The synthesis of AFP is recovered upon malignant development of liver tumors, germinogenic teratoblastomas and certain other cases. In early stages of embryonic development, AFP replaces albumin as a transport vehicle for fatty acids and other small-molecular hydrophobic substances (Deutsch H. F., 1991, Adv. Canc. Res. 56, 253-312).

Human AFP is a glycoprotein consisting of 590 amino acids and comprising about 4% of a carbohydrate component. The carbohydrate component is variable along with the maturation of the organism and tissue. The carbohydrate part affects structural, as well as the antigenic properties of AFP. Recombinant AFPs either are not glycosylated or glycosylation differs from natural human AFPs (Dudich et al. 2012 Prot. Expr. Purif., 84: 94-107). Methods for producing recombinant human AFP and its drug binding properties are described in U.S. Pat. No. 7,910,327 and US Patent Application 2011/0159112 A1 (Dudich E., et al.).

AFP can be selectively internalized by cells expressing specific AFP receptors (AFPRs), such as embryonic cells, mesenchymal stem cells, activated immune cells, cancer cells, or cells transformed by certain types of retroviruses (Mizejewski, 2001, Exp. Biol. Med. 226(5):377-408). Normal mature cells do not express specific AFPRs. This forms the bases to various applications of using AFP as a specific transporter of drugs to cells expressing AFPRs and to achieve extremely effective drugs with little influence on normal cells. AFP also may show synergistic effects to effector/drug molecules which it carriers. The use of AFP as a carrier of drugs is hampered by the fact that the AFP must not be immunogenic while should not be isolated from human or animal sources. It has been shown earlier that human AFP can be expressed, for example, in yeast cells and that such human recombinant AFP, hAFP, has biological properties related, but not identical, to native human AFP (Dudich E. et al., U.S. Pat. No. 7,910,327; Dudich et al., 2012 Prot. Expr. Purif. 84: 94-107). It was shown that rhAFP acts as a non-covalent carrier of various active poorly water-soluble ingredients aimed at regulating cells expressing AFPRs (Dudich E. et al., US patent application 2011/0159112 A1).

Interactions of pharmaceutical drugs with serum constituents are an important issue in the drug delivery (Kratz et al., J. Control. Release 2012; 157: 4-28). A general difficulty in formulation of drugs, especially injectable forms of them, is the fact that many of them are poorly water-soluble. Basically two approaches exist to solve the solubility problem, organic water-miscible solvents, or detergents and complexation with hydrophilic or amphiphilic macromolecules. The former approach has drawbacks of more or less poisonous additives, like oils, DMSO, or ethanol, since during the injection the solvents can make harmful high local concentrations before diluting into the blood stream, or to form non-homogenous aggregates. Examples on the complexation approach are synthetic copolymers, natural or recombinant proteins and large carbohydrates, like microbial dextrans. The term complex and complexation are used here in their usual meanings, i.e. for non-covalent binding to a carrier or formation of multimolecular complex. Sometimes active ingredients are bound covalently to carrier molecules for the aim at obtaining prolonged (slow release) indications but such drugs are distinctly different from the present invention. Different drug components (carriers, active ingredients, and additives) are metabolized and/or excreted with different mechanisms.

Proteins albumin and transferrin have attracted the most interest as drug carriers in the past two decades (Kratz, Control. Release 2008; 132:171-183). The carrier proteins can be classified: (1) selective carriers which can specifically bind to cells via membrane receptors, and (2), unselective carriers operating by utilizing a mechanism of passive targeting. Humanized antibodies and AFP exemplify class (1) carrier molecules. A severe drawback with protein drugs in general, especially with proteins without glycosylation or with non-native glycosylation, is their low stability against the catabolic pathways of foreign molecules in physiological fluids. A drug may quickly lose its activity, neutralized, and be secreted from body circulation while producing more or less harmful waste metabolites.

A universal problem with recombinant proteins is that they often are unstable, insoluble, or only partially soluble when expressed heterologously as known from standard textbooks in biochemistry. Mammalian proteins are often difficult to express in lower organisms. The host may lack appropriate source of cofactors, ligands, or there is no suitable environment for folding or post-translational modifications. Even if the expressed proteins are basically in a right conformation, they may exist in a loose “molten globule” conformational transition stage (loosely packed) form which is very sensitive to denaturation and attack by enzymes. The presence of certain ligands sometimes helps in obtaining structured conformation close to native protein but finding of proper conditions and ligands is a trial and error approach. If found proper stabilizing methods, compactness of a recombinant protein effectively prevents protein denaturation thereby increasing the half-life of the protein in an organism. For a drug carrier protein, its stability is of utmost importance.

The present invention was aimed at stabilizing human AFP, especially its recombinant form (rhAFP) to improve its operational life-time in physiological liquids. We found in the present invention that rhAFP, when completely released from bound ligands can form tight non-covalent complexes with metal ions. Various transition metals, such as Ni, Zn, Cu, Co, Fe, Pt are known to form complexes with any proteins including native AFPs (Mizejewski, 2001, Exp. Biol. Med. 226(5):377-408). Some of these useful metals are sometimes classified to belong to post transition metal series. However, the stiffening and biological functional effects of the metal complexes with AFP have not been realized and exploited in the prior art. Publication Permyakov, S. et al Biochim Biophys Acta 1586 (2002) 1-10 describes zinc-loaded AFP but is silent on the stabilizing or biological effects of the non-covalently bound zinc ions. On the contrary, this publication claims that tight cation binding is not accompanied with changes in protein structure and stability (shown in FIG. 6 in the cited reference). US patent application publication US 2010/0022442 A1 (Tsai, M. H) does not include observations on the stabilizing effects of zinc on non-fragmented proteins.

Documents WO2007056852 A1 (Constab Pharmaceuticals), US 2011159112 A1 (Dudich et al.) and WO2010129337 A2 (Ceramoptec Industries) describe pharmaceutical compositions with AFP but do not disclose compositions containing AFP stabilized with non-covalently bound transition or post transition metal ions.

The recombinant forms of AFP especially benefit from the complexation with metal ions since it was shown in the present invention that metal ions harden or stiffen the loosely packed recombinant protein AFP. Furthermore, the complexation of rhAFP with metal ions may twist the protein into a more stable conformation. However, the metal ion-induced conformational change did not alter rhAFP's ability to form complexes with a number of drugs which bind the metal-free rhAFP in the prior art (Dudich E. et al., US patent application 2011/0159112 A1). The small-molecular hydrophobic ligands further stabilized protein structure. The terms complex and complexation (the act of forming a complex or adduct) are used here in their usual meanings, i.e. for non-covalent binding of metal ions or drugs to AFP through hydrophobic or other types of non-covalent interactions. A special new feature of the present invention is the design of metal ion/rhAFP/drug complexes which are principally different from those features obtained by chemical binding resulting in formation of protein/drug conjugates described by Feldman et al., 2000, Biochemistry (Moscow); 65:967-971. While in the present invention it was demonstrated a significant improvement in stabilities of the metal ion-rhAFP-drug molecular complexes, they could carry out the biological functions of the native AFPs. The preferred low-toxic metal ion, Zn++, had positive synergistic biological effect in certain applications to AFP and to AFP-complexed drugs.

The compositions are exemplified in detail with the Zn(II) ion-liganded rhAFP carrying small-molecular anticancer drugs like paclitaxel, curcumin, resveratrol, genistein, lycopene, doxorubicin, etoposide, and cisplatin or their mixtures. They employ different mechanisms of action for treatment of different cancers. A synergism of biological effects exists between AFP, metal ions, and toxins. Pharmaceutical compositions according to the present invention are intended for use in targeting therapy especially in oncology. The present invention overcomes several drawbacks of previously existing formulations for cancer medicines. These and other advantages of the invention, as well as additional inventive features, will be obvious from the description of the invention provided herein.

### BRIEF SUMMARY OF THE INVENTION

The objective technical problem of the present invention to be solved was how to improve the targeted drug delivery comprising AFP. The problem was solved by loading the carrier AFP with non-covalently bound transition or post transition metal ions. The present invention therefore relates to significantly better, still pharmaceutically acceptable, formulations of drugs. The metal-stabilized AFP employs specific interactions with the AFP receptors (AFPRs) allowing targeting delivery to AFPR-expressing cells. The first embodiment of the invention is that the structure of human recombinant AFP liganded to metal ions and various drug molecules is converted into a stable conformation, whereas this complex still efficiently targets AFPR-expressing cells. The improved stability was unambiguously shown here by physical measurements, and the improved targeting and biological effects were confirmed by experimental data. Positive effects of the metal stabilization of AFP as a drug carrier were thoroughly exemplified with two well-known cancer drugs, paclitaxel and curcumin, which target to AFPR-expressing cells due to their binding to AFP, while these drugs have completely different mechanisms of action. Other examples are shown to illustrate the universal value of the invention for targeting therapy.

### BRIEF DESCRIPTION OF THE FIGURES

The following figures and drawings illustrate the present subject matters of the invention:

FIG. 1A shows the cytotoxic effect of rhAFP and Zn-functionalized rhAFP (Zn-rhAFP) for the breast carcinoma cell line MCF-7 in vitro as it was assessed by the [H³]-thymidine incorporation assay. The mean data from three independent experiments±SD (mean square deviation) are shown.

FIG. 1B shows that Zn-rhAFP abrogates cytotoxic effects of Ptx in normal mice splenocytes. Various doses of paclitaxel (Ptx), Zn-rhAFP and their equimolar combination Ptx/Zn-rhAFP were tested for the unspecific toxicity against normal CBA mice splenocytes in vitro. Splenocytes were incubated for 48 h with various doses of Zn-rhAFP, Ptx or equimolar Zn-rhAFP/Ptx 1:1 complex. Zn-rhAFP was prepared in a molecular ratio Zn:rhAFP=4:1. Viable cells were visualized by trypan blue staining. The data are expressed as percentage of viable cells in experimental cell cultures relative to untreated control. Mean of three independent experiments is shown.

FIG. 2 shows the cytotoxic effect of rhAFP/Ptx combination as compared to paclitaxel (Ptx) or Zn-rhAFP standalone for the breast carcinoma MCF-7 (A), rat glioma C6 (B) and human hepatoma HepG2 (C) cell lines as it was assessed by colorimetric MTS cell viability assay at the incubation time of 48 h. Inhibition of cell viability by Zn-rhAFP; Ptx; and Zn-rhAFP/Ptx composition in molecular ratios Zn-rhAFP:Ptx=1:1, 1:3, and 1:5 was studied. Effective concentration of combined Zn-rhAFP/Ptx was monitored by Ptx concentration. Zn-rhAFP was prepared in molecular ratio Zn:rhAFP=4:1.

FIG. 3 shows photomicrograph of stained with DAPI control MCF-7 cells and those treated with paclitaxel (Ptx), rhAFP, and rhAFP+Ptx. Concentrations of the compounds were: rhAFP: 3.0 μM, Ptx: 10 μM. Incubation time was 48 h. Patterns on the top (A): magnification ×100; bottom (B): magnification ×400. Cell nuclei were stained with DAPI and visualized with a fluorescent microscope Axioplan, Zeiss equipped with corresponding color filter kits. Apoptotic nuclei at s panel B are shown by arrows. Panel C shows the change of relative cell counts due to the treatments. Total number of cells in the control wells was taken as 100%. The average data for five wells±mean square deviation are shown.

FIG. 4 shows the cytotoxic effect of rhAFP/Cur combination as compared to curcumin (Cur) standalone for Burkitt lymphoma Raji (A, B), as it was assessed by H3-thymidine incorporation assay at colorimetric MTS cell viability assay at the incubation time of 24 h. Inhibition of cell proliferation by various doses of curcumin and Zn-rhAFP/Cur composition was studied. Zn-rhAFP was prepared in molecular ratio Zn:rhAFP=4:1.

FIG. 5 shows the cytotoxic effect of rhAFP/Cur combination as compared to curcumin (Cur) standalone for breast carcinoma MCF-7 (A), and hepatoma HepG2 (B), as it was assessed by colorimetric MTS cell viability assay at the incubation time of 48 h. Inhibition of cell viability by various doses of curcumin and Zn-rhAFP/Cur composition was studied. Zn-rhAFP was prepared in molecular ratio Zn:rhAFP=4:1.

FIG. 6 shows combined anti-tumor efficacy of Zn-rhAFP (3.0 μM) and paclitaxel (100 nM) for various types of tumor cell lines in vitro: human mammary carcinoma MCF-7; human hepatoblastoma HepG2 and rat glioma C6 (A). Combined tumor-suppressive efficacy of Zn-rhAFP and various anti-tumor compounds against mammary breast carcinoma cells MCF-7 (B): Ptx, paclitaxel (100 nM); Lyc, lycopene (0.5 μM); Cur, curcumin (0.5 μM); Gst, genistein (1.0 μM); Res, resveratrol (0.5 μM); Dox, doxorubicin (10 nM); Cis, cisplatin (20 nM) and Eto, etoposide (20 nM). Concentration of Zn/rhAFP was 3.0 μM, molecular ratio of Zn:rhAFP was 4:1. Cells were incubated with combination of rhAFP/medicine for 48 hrs and thereafter assessed for cytotoxic effect by MTS assay.

FIG. 7 shows the effect of rhAFP and curcumin standalone or in combination on the paclitaxel-induced NF-κB activation and its nuclear translocation in mammary carcinoma MCF-7 cells. The cells were incubated with paclitaxel (10 μM) with addition of Zn-rhAFP (3.0 μM) and/or curcumin (3.0 μM) for 2 h and then assessed for localization of p65 by immunocytochemistry. Activated NF-μB (p65) was detected by treatment of the cells with phospho NF-μB p65 rabbit polyclonal antibody (Cell signaling technologies, USA) followed by secondary goat anti-rabbit Ab-Alexa594 red (Molecular Probes, USA). Cells were visualized at magnification ×400 by fluorescent microscope Axioplan; Zeiss (Germany) equipped with corresponding color filter kits.

FIG. 8 shows distribution of the activated p65 between cytosol and nucleus. The relative amount of p65 in the cytosol and nucleus of the cells was assessed by ImageJ software and was taken as 100% in the control (cytosol+nucleus). Total amount of activated p65 in control untreated cells was taken as 100%. The experimental data are represented as percentage of control±SD from three independent calculations in various observation fields.

FIG. 9 shows the notable enhance of endocytosis of curcumin in the presence of Zn-rhAFP in the nuclei of human mammary carcinoma MCF-7 cells. Left panel: 0.1 μM curcumin alone; right panel: 0.1 μM curcumin in the presence of 1.4 μM Zn-rhAFP. The curcumin's own green fluorescence was detected by fluorescent microscope Axioplan; Zeiss (Germany) equipped with corresponding color filter kits in a magnification of ×400.

FIG. 10 proves the formation of multimolecular complexes between rhAFP, Zn ions, paclitaxel (A) and curcumin (B). The thermodynamic parameters describe the changes of the tertiary structure of rhAFP due to ligand binding as it was measured with the adiabatic scanning microcalorimetry technique. The excess heat capacity function Cp,exc(T) was calculated for the untreated ligand-free rhAFP, rhAFP loaded with Zn ions (A, B), and Zn-rhAFP loaded with paclitaxel (A) or curcumin (B). The protein concentration was 2.0 mg/ml in PBS, pH 7.4. Relative amount of ligands were: Zn/rhAFP=4/1; Ptx/rhAFP=3/1; Cur/rhAFP=3:1.

### DETAILED DESCRIPTION OF THE INVENTION

The invention provides basic biologically-established components for designing significantly improved delivery mechanisms for drugs of common human diseases, especially for cancers, which are becoming the highest mortality factors preventing the increase of average life-span of humans. The invention exploits recombinantly expressed protein, resembling human embryonic serum carrier protein, alpha-fetoprotein (AFP) as a specific transporter or carrier of drugs. AFP is highly soluble protein like its analogue serum albumin. Both of them, however, contain embedded hydrophobic pockets or clefts which can bind hydrophobic molecules. Therefore AFP, like albumin, suits for drug delivery purposes. Recombinant human AFP is practically the only possible alternative for those purposes for ethical and medical reasons. The important difference between AFP and albumin is the fact that they employ very different delivery mechanisms: AFP is selective whereas albumin is unselective carrier in entering cells. AFP is internalized by certain types of undifferentiated cells, which are able to express specific membrane AFPRs (stem cells, tumor cells, activated immune cells), whereas albumin can be taken up by all of the cell types. This feature makes AFP as a superior carrier for targeted cancer therapy.

Whether recombinant AFP is used as a carrier for injected drugs, in particular, its structure must closely resemble the native AFP. The human organism should accept the injected protein as belonging to the category of familiar proteins so that organism's defense systems will not be alarmed. RhAFP's structure must also be native enough so that cell AFPRs will recognize it and function in a desired way. Recombinant proteins have a structural uncertainty originated from the glycosylation mode provided by expression organisms and also due to other different mechanisms of post-translational modifications existing in mammalian and microbial host cells. Natural AFP contains 4% of carbohydrates by weight located on the protein surface which affects to its immunogenicity, biochemical stability, as well as probably to binding to AFP receptors (AFPRs). It was found previously, that human AFP produced in a yeast host could be an effective carrier of drugs and can bind various hydrophobic ligands (Dudich E. et al., US patent publication 2011/0159112 A1). Although recombinant human AFP (rhAFP) expressed in yeast host was employed in the present invention, it not limited to yeast rhAFP only. It can be expressed also in many other eukaryotic and prokaryotic organisms and even by human cell lines producing correctly post-translationally modified AFP. Equally well, isolated human AFP may be used in some applications. Furthermore, certain limited variations in the primary sequence of human AFP may be done in the present invention, especially by using rationally designed mutations in the 3-D structure of AFP, without any significant loss of the physiological properties of AFP and in its drug formulations.

According to the present invention AFP carrier can be effectively stabilized with metal ions to get significantly better carrier properties over the prior art. The protein flexibility will be thereby hardened and AFP becomes resistant to decomposition. However, this stabilization by hardening did not affect negatively to its biological functions. Metal ions obviously bind to AFP as non-covalent chelate complexes. The affinity of metal ions to form the complex with proteins is well known and it depends on the charge, ionic radius, the degree of hydration, as well as orbital geometry (Metzler, D., Biochemistry, 2001, p. 311; Academic Press, New York). The binding strengths of various metals follow rules which predict that chelate stabilities increasing in the first transition metal series from Mn(II) to Zn(II) ions.

To prepare metal and drug-loaded forms of AFP, native or recombinantly produced AFPs must be first deionized and defatted to form a de-liganded AFP by removing existing non-covalently bound metal ions and hydrophobic molecules. This procedure will provide maximal control of the product that is important for pharmaceuticals. The defatting methods should be as gentle as possible to conserve nativity of protein structure because in vitro refolding of AFP appeared to be extremely difficult. The present invention mainly exploited charcoal/HCl treatment at pH 3.0 to avoid protein denaturation (Example 2).

The present invention describes designs of metal-stabilized AFPs and their uses for medical and pharmaceutical purposes. While quite many metals may be used for stabilizing recombinant AFPs including di- and polyvalent metal ions like Fe, Ni, Co, Mn, Mg, Ca, Al, Pt, and Cu, only due to its low toxicity and common use in medicine, Zn(II) ions were found preferable for most of the objectives of the present invention. Zinc is a natural element needed in human physiology being found, for example, in an important role in cell nuclear compartments liganded with Zn-finger proteins. In amounts used, according to the present invention liganded with rhAFP, Zn ions are not toxic to humans. Moreover, depending on the stability requirement of rhAFP, different stoichiometric compositions of Zn ions with rhAFP can be prepared. An advantageous method of preparing metal complexes with the recombinant proteins is adding the desired metal salt into growth medium of the pro- or eukaryotic host cell cultures. In such cultivations the expressed rhAFP will be stabilized and give higher yields. This observation further confirms that metal ions stabilize rhAFP structure in physiological conditions. A mixture of different metal ions might be useful to be bound to rhAFP to lower the toxic risk of one metal. If the delivery mechanism is very specific, like with AFP, it is possible to apply toxic metal ions not found in normal human physiology for targeting delivery with therapeutic goals.

The metal ion-hardened or stabilized structures of rhAFPs were further bound to various drugs or pro-drugs to improve their bioavailability, solubility and targeting properties. The drugs described earlier (Dudich E. et al., US patent publication 2011/0159112 A1) were successfully bound to metal ions-stabilized AFPs with different stoichiometric combinations. Since AFP has various hydrophobic binding pockets with different sizes and chemical properties, the spectrum of drugs to be bound to AFP-metal complexes is wide. The drugs predominantly bind to AFP with hydrophobic and Van Der Waals forces. This is a favorable coincidence since majority of drugs are poorly water-soluble (hydrophobic) and therefore bind tightly to AFP's binding pockets. The number and properties of the AFP's binding pockets cannot be forecasted and therefore the binding stoichiometry for a drug molecule depends on unknown structural details. For a relatively hydrophobic drug, always at least 1-10 (high to moderate) binding pockets should be found in an AFP molecule. Normally, the metal and drug binding are independent, but in certain cases drug and metal binding had synergistic mechanisms producing cumulative stabilizing effects onto AFP. It is obvious that any small hydrophobic bioactive molecule can bind into metal-stabilized AFP also from biological fluids and not only in specially prepared solutions in vitro, while the unique carrier properties of AFP remain. Therefore, there may be needed to administer stabilized AFP and the drug or drug mixture separately and allow the components bind in natural environment of the human body.

Whereas the pharmaceutical industry often avoids multidrug formulations because of extra legal regulatory impacts, we have demonstrated in the present invention that two or more different drugs can be loaded simultaneously to a metal-stabilized rhAFP. In specific conditions it may be valuable to have two or more different drugs to be transported into target cells in the certain stoichiometric ratios. For example, Zn-rhAFP was loaded with both paclitaxel and curcumin. Paclitaxel is a widely used anti-cancer toxin whereas curcumin is a known sensitizer of cells to cancer drugs.

Different pharmaceutical final forms, containing necessary additives, can be designed based on the metal-stabilized AFPs of the present invention. The drug can be in liquid or dry form and can be administrated in different ways. The pharmaceutically acceptable protein carrier comprises AFP, preferably human recombinant alpha-fetoprotein (rhAFP) that is preferably Zn- or Fe-functionalized. Recombinant hAFP suitable to the present invention may be glycosylated or non-glycosylated. The defatted Zn-rhAFP can be loaded with a drug exemplified by paclitaxel (Ptx) and/or curcumin in a solution including organic solvents or detergents in necessary amounts needed at least to a partial solubilization in water. The organic solvents may be partly or completely removed in the final drug form. Structurally protein-bound water can be removed from metal-AFP complex almost totally without collapse of the protein structure which again increase the storability of the pharmaceutical products. Zn-rhAFP-based paclitaxel formulations might be prepared to contain one rhAFP molecule with 1-10 of Ptx and 1-10 of Zn molecules (equivalent to their molar ratios). It is also possible to prepare nanoparticles of molecular size of 120-200 nm comprising several (5-20) rhAFP molecules, which are loaded with Zn and paclitaxel containing 5-100 molecules of each ligand per one rhAFP-based nanoparticle. The technical processes for such nanoparticles are known in prior art in the context of albumin. Nanoparticle format should be considered to be only an extension of the present invention involving metal ion stabilized rhAFP. Without stabilization recombinant AFPs would denaturate and aggregate during the nanoparticle preparation process.

The invention also provides means exploiting metal-stabilized AFP for delivery of a pharmaceutical agent selectively into tumor cell avoiding healthy cells, methods for sensitization of tumors to chemotherapy by blocking apoptosis resistance, and methods of reducing of adverse side effects of administration of pharmaceutical compositions. The combination of Zn-rhAFP and paclitaxel can be straightforwardly used to treat breast and brain tumors and inhibit their metastases.

We have developed a recombinant version of human alpha-fetoprotein expressed in the yeast strain S. cerevisae with transfected human AFP gene (see U.S. Pat. No. 7,910,327; USA patent application 20110159112; EP No 1807518; Eurasia patent No 011606) and yeast strain S. cerevisae with multiple chromosome-integrated synthetic human AFP genes expressing both glycosylated and non-glycosylated rhAFP species (Dudich et al., Prot. Expr. Purif., 2012; 89:94-107). The Zn-rhAFP, used in the present invention, is structurally and functionally different from that described in U.S. Pat. No. 7,910,327 and in (Dudich et al., Prot. Expr. Purif., 2012; 89:94-107), because it was functionalized with metal ions giving a novel structural form and novel properties to rhAFP (Me-rhAFP), differing by its tertiary structure and functional activity. This was shown by the increased stability against heat denaturation as well as to destruction with chemical and biological agents. Three-dimensional X-ray structural data are not available for AFP and the conformational changes were monitored here by circular dichroism and differential adiabatic scanning microcalorimetry techniques that are the best available methods presently. The differences in the conformation between Zn-rhAFP and rhAFP resulted in change of the functional activity as described in the Example 3, showing notable increase in tumor-suppressive activity. Non-covalent binding of metal ions induced notable conformational change in the tertiary structure of the rhAFP molecule leading to the structural stabilization of rhAFP molecule and hence prolonged operational life span in biological liquids and increased storage stability. Simultaneously, loading of the rhAFP with metal ions led to a significant increase in the pro-apoptotic activity and therapeutic efficacy of the Me-rhAFP as compared to the rhAFP. Therefore, metal-functionalized rhAFP represents a novel molecular drug carrier, which has structure and functional activity distinctly different from rhAFP, and hence Me-AFP can serve as a significantly better drug carrier over non-functionalized rhAFP.

We showed in the present invention that Zn-rhAFP effectively transported drugs inside different types of tumor cells and thus provided a targeting tumor-specific delivery of this cytotoxic compound. AFP represents a family of carrier serum proteins that have an inherent function to bind certain small hydrophobic drug molecules and deliver them into selected types of developing cells, including tumor cells, activated immune cells, and stem cells, all of which are capable of expressing specific AFPRs. Other examples with other metal complexes of AFP and with other drug molecules can be used to conclude that metal complexes of AFP form a specific universal delivery system. Since the metal and the drug, at least in most cases, appeared to bind independently of each others to AFP, the binding studies of drugs to AFP in the prior art can be considered to bind also to Me-stabilized AFP. Whereas AFP itself has several advantages as carrier in the drug delivery systems, the Me-AFP reinforces all those below-listed advantages:

(1) Solubilization of the hydrophobic drug in aqueous solution without any contamination from toxic solvents;

(2) Possibility of easy sterilization;

(3) Possibility of lyophilization with further reconstitution without loss of activity;

(4) Targeting delivery of pharmaceutical composition in AFPR-expressing cells;

(5) Significant reduction of the side effects associated with non-specific systems;

(6) AFP-mediated sensitization of chemoresistant tumors to weak apoptosis signals by blocking of XIAP-dependent inhibitory signaling and NF-κB activation;

(7) Recombinant human AFP expressed in yeasts does not have risks connected to proteins obtained from human serum.

The technical solutions most closely related to the present invention are pharmaceutical formulations comprising various types of non-covalent complexes of albumin and cancer drugs exemplified by paclitaxel (see, e.g., U.S. Pat. Nos. 7,820,788; 7,923,536; 7,758,891; 7,780,984; 6,310,039).

The general functional difference between albumin and metal ion-stabilized or non-stabilized AFPs is the existence of tumor-specific membrane AFPRs allowing tumor-selective binding of AFP-drug complexes with further internalization inside tumor cells avoiding normal healthy cells lacking the AFPRs. Thus, AFP as a carrier protein to deliver toxic compounds to tumor cells provides active targeting whereas albumin as a carrier protein provides passive targeting.

The use of AFP—with paclitaxel formulation will allow reducing or elimination of the side effects associated with the parenteral or in vivo administration of traditional formulations that will provide a vehicle for targeting paclitaxel selectively to cancer cells avoiding normal tissues. There is also a need for a pharmaceutical composition that is sterile, and methods of preparing such a composition. In addition, there is a need for a pharmaceutical composition and method that reduce or eliminate oxidation of pharmaceutical formulations to prevent drug destabilization.

As known, AFP operates not only as a simple mechanistic carrier but simultaneously functions as an active tumor suppressive agent. It was demonstrated that AFP can sensitize tumor cells to the apoptotic stimuli induced by other factors operating by blocking inhibitory signaling by inhibitor of apoptosis proteins (XIAP, cIAP2) and also by blocking of nuclear transcription factor NF-μB activation induced by cytotoxic drugs in tumor cells (Dudich et al., 2006; FEBS J. 273: 3837-3849). We show here that Zn-rhAFP blocks the paclitaxel-induced activation of NF-κB and its nuclear translocation leading to the increase in chemosensitivity to paclitaxel and enhanced apoptosis. According to the present invention Me-stabilized rhAFP functions by similar ways but is more effective than rhAFP alone.

Targeting effect of the covalently attached cytotoxic drugs to human embryonic AFP or to the recombinant fragment of AFP were described with covalently attached doxorubicin (Feldman et al., Biochemistry (Moscow) 2000; 65:967-971) or paclitaxel that was introduced to nanosomes with attached recombinant chimeric domain (a part which was believed to be targeting unit of AFP molecule) originated from the human AFP sequence (Godovannyi et al., 2012 Nanotechnologies in Russia, 2012, Vol. 7: 76-84). These experiments do not, however, provide basis for successful drug formulations since: (1) covalent chemical binding of a drug induces conformational changes in the structure of the carrier AFP molecule which will induce harmful immunological reactions; (2) the in vivo use of cytotoxic drugs conjugated to antibodies (antibiotics, antimetabolites, bacterial toxins) has also been impeded by such obstacles as immune reactions toward such conjugates and their rapid clearance rates imposed by Kupfer cells of the liver (Mizejewski, Exp. Anticancer Therapy, 2002; 2(6): 709-735), (3) covalently bound toxin does not detach from AFP inside or in the vicinity of cancer cells, (4) chimeric domains will not be transported naturally inside the cancer cells. The present invention, exploits only entire recombinant AFP molecules stabilized with metal ions that form a novel significantly improved drug delivery vehicle. It is shown in the present invention that true non-covalent molecular complexes are formed.

Our data obtained for recombinant yeast-derived human rhAFP have demonstrated that binding of metal ions was accompanied with a significant conformational change in the tertiary structure of rhAFP resulting in stabilization of the molecule against heat denaturation (Example 9). Simultaneously, functional activity of the Zn-functionalized rhAFP was notably improved as compared to the ligand-free rhAFP molecule (Example 3). The data described in the present invention show that metal ion-functionalized rhAFP represents a novel structural form of the macromolecule differing from the parent rhAFP.

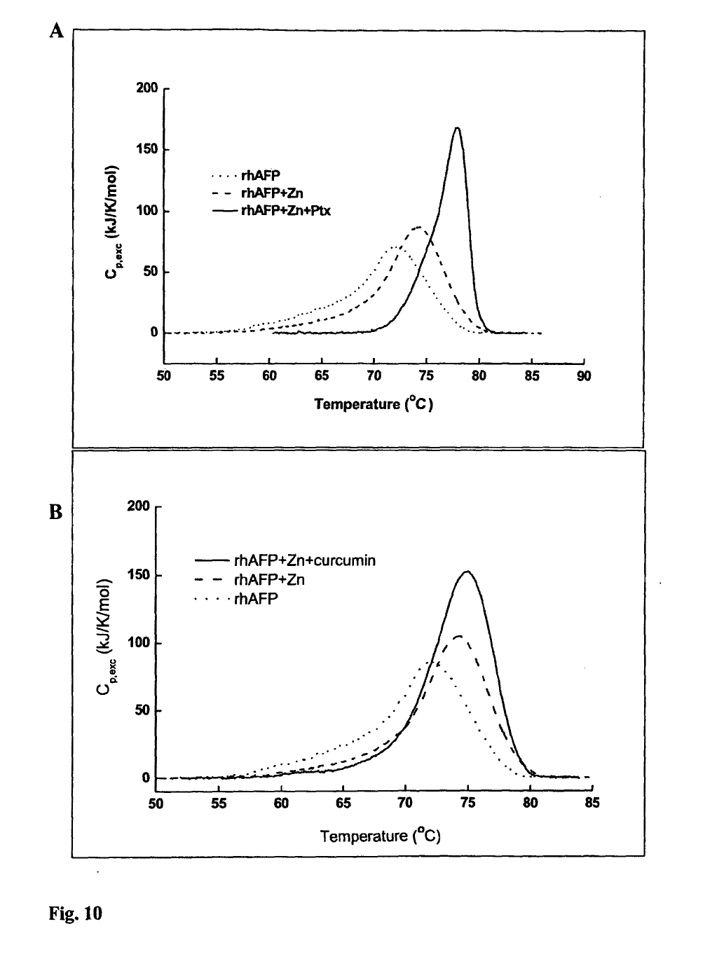

The effect of stabilization of the fetal AFP molecule by organic ligands binding was reported by Uversky et al., Biochemistry 1997; 36: 13638-13645. Binding of metals Cu(II), Zn(II) was documented (Permyakov, et al., Biochim. Biophys. Acta. 2002; 1586:1-10), but the effect of structural stabilization of AFP for its practical significance was not realized. Loading with natural ligands of recombinant proteins obtained by heterologous expression in the foreign host cells, would be desirable to maintain nativity and stability of the general protein conformation providing simultaneously resistance to enzymatic cleavage and protein degradation. Our experimental data (FIG. 10) show that rhAFP requires hydrophobic ligand binding to stabilize its tertiary structure. Adiabatic scanning microcalorimetry experiments demonstrated that binding of metal ions and paclitaxel were resulted in a significant synergistic stabilization showing the formation of the macromolecular complex Me-rhAFP/Ptx. Similar results were obtained with curcumin complexes. Moreover, simultaneously a significant increase in biological activity was demonstrated as compared to the same doses of Ptx or curcumin standalone.

AFP-mediated tumor-suppressive activity was reported by various authors (Bennett et al., Breast Cancer Res. Treat. 1997:169-179; Dudich et al., Eur. J. Biochem. 1999; 266:750-761). It was shown to be related to apoptosis (Semenkova et al., Eur. J. Biochem. 2003; 70: 4388-4399). The experimental data demonstrated that various species of AFP including natural serum embryonic eAFP and also yeast-derived recombinant rhAFP operate by blocking inhibitory signaling induced by direct binding to intracellular inhibitor of apoptosis proteins (XIAP). This leads to the activation of apoptosis response to weak stimuli in tumor cells with significant sensitization to chemotherapeutic drugs (Dudich, E., et al., FEBS J. 2006; 273: 3837-3849; USA patent application No 20110159112).

Our experimental data obtained during the last decade strongly argument that both natural serum embryonic eAFP and also recombinant rhAFP mediate triggering of apoptosis selectively in tumor cells by employing double-targeting strategy: (1) specific penetration into tumor cells via membrane AFP-receptors and (2) subsequent blocking of XIAP that is constitutively over-expressed in tumor cells and that primarily determines cells resistance to apoptosis induced by chemotherapeutic drugs (Dudich et al., FEBS J. 2006; 2733837-3849: USA patent application No 20110159112); (3) the experimental data presented in the present invention showed that Zn-rhAFP sensitize tumor cells to drug-induced cytotoxicity by blocking NF-μB activation and its nuclear translocation (Example 8). The tumor-selectivity of AFP's pro-apoptotic activity together with AFP's positive modulation of the apoptotic signals mediated by other factors are believed to be potent cancer therapeutics (Dudich, et al., USA patent application No 20110159112). Taking into account high therapeutic potential of the AFP-based drugs in treatment of cancer and autoimmune diseases we have previously developed a technology of the high-yield production of rhAFP. The yeast-derived rhAFP has similar functional and structural characteristics as those obtained from human cord serum (Dudich et al., Prot. Expr. Purif. 2012; 84: 94-107).

Our experimental data shown here demonstrate that paclitaxel binds rhAFP by forming high affinity non-covalent complex via a mechanism of hydrophobic interaction between the molecules allowing drug solubilization and its targeting delivery to cancer cells avoiding normal cells. Paclitaxel-loaded rhAFP markedly increases water solubility, bioavailability and cytotoxicity of the drug against resistant human breast cancer cell lines and rat glioma cell lines in vitro. AFP-receptor targeting delivery of the Zn-rhAFP/Ptx markedly amplifies cytotoxicity toward cancer cells and decreases unspecific side toxicity. Zn-rhAFP/Ptx complex is soluble in water and can be used for preparation of injectable forms. The similar experimental data had been obtained for Zn-rhAFP/curcumin complexes and also a number of other tumor suppressive pharmaceutical agents involved in non-covalent interactions with human AFP. In this invention synergistic effects had been shown for pharmaceutical compositions comprising non-covalent complexes of Zn-rhAFP with curcumin, resveratrol, genistein, lycopene, doxorubicin, cisplatin, and etoposide (Example 7).

Me-rhAFP/drug complexes are superior over the non-stabilized AFP drug complexes as shown in the present invention. The advantages of the novel drug delivery system based on Me-stabilized AFP forms a definitive improvement over the prior art. Since cancer is extremely common cause for death, presently and especially in the future, the present invention is predominantly, but not limited to, be used in designing various types of cancer drug delivery systems. The invention will be further illustrated by the following non-limiting Examples.

### Example 1

### Expression of Recombinant Human rhAFP in Yeast. Purification and Characterization of Various Human AFP Species

Glycosylated wild-type form of recombinant human AFP (rhAFP) was obtained from the culture medium of recombinant yeast strain Saccharomyces cerevisiae YBS723/pKX-AFP secreting rhAFP in the culture medium as described in (U.S. Pat. No. 7,910,327). Sugar-free non-glycosylated mutant rhAFP₀ was expressed in the yeast strain S. cerevisiae with multiple chromosome-integrated synthetic human mutant AFPₘᵤₜ gene with single point mutation of the N-glycosylation site (N233S) able of secreting of protein product in cultural liquid as described in (Dudich et al., Prot. Expr. Purif. 2012; 84: 94-107). Pilot scale high cell-density fermentation of both producer strains was carried out in the YPDGE medium (1% yeast extract, 2% peptone, 2% glucose, 1.5% glycerin, 0.1 M K₂HPO₄+KH₂PO₄, pH 7.0) in a 2-L Biostat B bioreactor (B. Braun Biotech International, Germany). The fermentation was carried out at 30° C. and pH 7.0 (automatic maintenance). The content of the dissolved oxygen was maintained >30%. The 150 mL of yeast cell culture (OD₆₀₀=15) were inoculated in 1 L of YPDGE medium with addition of leucine (30 μg/ml) and propagated for 72 hours at 30° C. During fermentation the replenishment with YPD medium (5% yeast extract, 10% peptone, 25% glucose) was continuously performed. After achievement of OD₆₀₀, equal to 280 optical units, the content of rhAFP (rhAFP₀) in the CL was analyzed. The relative and total content of the rhAFP or rhAFP₀ in the CL of high density cultures of yeast producer strains were determined by ELISA and SDS-PAGE.

Both rhAFP species were isolated from the cultural liquid by using simple and effective isolation procedure employing three stages: (1) cation-exchange chromatography on the CM-Sepharose FF (GE Healthcare, Germany) column; (2) anion-exchange chromatography on the DEAE-Sepharose FF column (GE Healthcare, Germany); and (3) gel chromatography on Sephacryl S-200 HR (Amersham Pharmacia, USA) as described (Dudich et al., Prot. Expr. Purif. 2012; 84: 94-107). Fetal serum human eAFP was isolated from the human cord serum as described previously (Dudich et al., Biochemistry 1999; 38:10406-14).

Finally, all the AFP preparations were filtered through a strong basic anion exchanger membrane system Sartobind® (Sartorius Stedim Biotech GmbH, Germany) to guarantee endotoxin clearance that is strongly required for medical use of biological products. It should be noted that endotoxins, which presence is typical for E. coli extracts, usually are not presented in the yeast extract. But we added this purification stage to avoid external endotoxin contamination. The final endotoxin contamination was determined to be <1.5 ng/mg as it was shown by Gel-clot LAL test (Lonza, Switzerland). To avoid endotoxin contamination all the buffer solutions have been prepared with the apyrogenic water using sterilized glass ware, and all the isolation procedures were produced in a laminar cabinet at 10° C.

Purity and homogeneity of all human AFP species was more than 98% as assessed by SDS-PAGE and Western blotting with anti-AFP rabbit polyclonal antibodies.

### Example 2

### Preparation of rhAFP Complexes with Ligands

Paclitaxel was obtained from Samyang Genex Corporation, Korea. Doxorubicin, etoposide, cisplatin, curcumin, genistein, resveratrol and lycopene as well as dimethyl sulfoxide (DMSO), tetrahydrofuran (THF) and other chemicals were purchased from Sigma (St.-Louis, USA) if not specified elsewhere and used as supplied.

Ligand-free defatted rhAFP was prepared by charcoal/HCl treatment, as described in (Dudich et al., Biochemistry 1999; 38: 10406-10414). Briefly, 100 mg of activated charcoal (Sigma) were added to a 30 mg sample of rhAFP in 20 ml of distilled water at 0° C. The pH was then carefully adjusted to 3.0 with 0.1 N HCl and the mixture incubated with shaking at 0° C. for 2 h. The solution was then centrifuged at 25,000×g for 30 min at this temperature. The supernatant containing the ligand-free protein was then decanted from the charcoal and adjusted to pH 7.0 with 0.1 N NaOH. Complexes of rhAFP with Zn-ions were prepared by addition of 4-fold molar excess of ZnCl₂ dissolved in phosphate buffered saline, pH 7.4 (PBS), followed by overnight dialysis against fresh PBS. RhAFP complexes with paclitaxel, curcumin, resveratrol, genistein doxorubicin, etoposide and cisplatin, were prepared by 2-h incubation of the protein solution in PBS (10 mg/ml) with corresponding amount of these ligands dissolved in DMSO or ethanol to keep protein:ligand ratio from 1:1 to 1:10. rhAFP/lycopene complexes were prepared by incubation of rhAFP with 2 mM stock solution of lycopene in THF. The rhAFP/ligand complexes were used for cell culture or spectroscopic and microcalorimetry experiments. The final concentration of polar solvents in cell culture did not exceed of 1-2% and was subtracted as a control in cell viability experiments. The unbound ligands were removed by dialysis before microcalorimetry experiments. Other metal ion complexes of rhAFP with or without drugs and active ingredients were prepared basically in the similar way.

### Example 3

### Study of the Tumor-Suppressive Activity of rhAFP and Zn-Functionalized rhAFP

Promega CellTiter 96 AQueous Non-Radioactive Cell Proliferation (MTS) assay was purchased from Promega (USA). Cell culture medium DMEM, fetal bovine serum (FBS) and antibiotics were supplied by Sigma (USA). Tumor cell lines MCF-7 were originated from American Type Culture Collection.

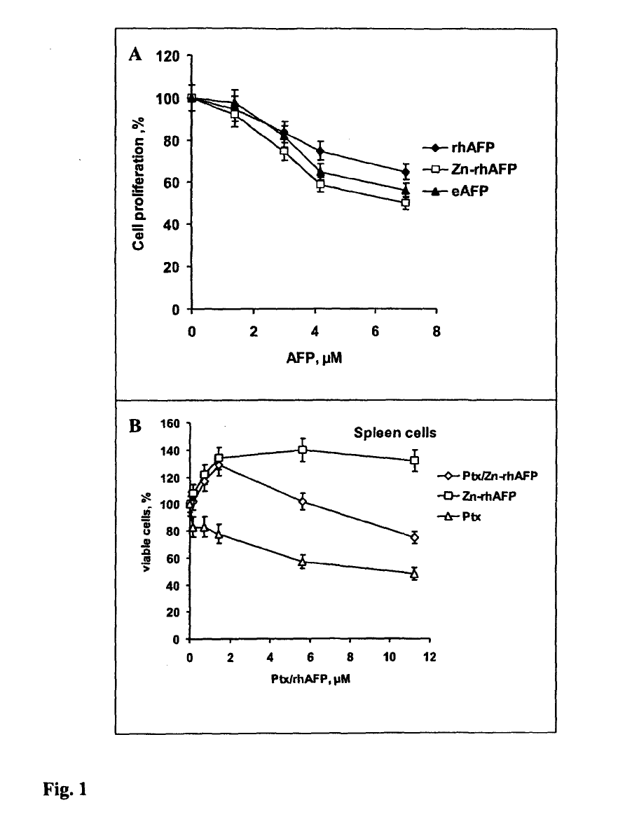

Effect of binding of Zn ions to rhAFP and sugar-free rhAFPo species was tested by measuring of their dose-dependent tumor-suppressive effect for mammary carcinoma cell line MCF-7 in vitro, as it was earlier described for natural embryonic eAFP (Dudich et al., Eur. J. Biochem. 1999; 266: 750-761). MCF-7 cells were incubated with various doses of rhAFP (rhAFPo), Zn-rhAFP (Zn-rhAFPo) or with natural embryonic eAFP for 48 h, and thereafter their viability was assessed by the colorimetric MTS assay (FIG. 1A). These data indicated that rhAFP revealed slightly less effective tumor suppressive effect as compared to the natural eAFP showing IC₅₀ of 3.5 μM for rhAFP and 3.0 μM for eAFP, whereas Zn-rhAFP showed a significant increase in tumor suppression activity and showed increase in IC₅₀ value reaching of 2.5 μM (Table 1).

Surprisingly, Zn-rhAFP showed enhanced tumor-suppressive activity as compared to its natural serum analogue eAFP. Similar results had been obtained for the non-glycosylated rhAFPo showing that Zn-binding induced a significant increase in its functional activity (data not shown). Therefore, binding of Zn ions, significantly improves tumor-suppressive activity of both rhAFP and rhAFPo.

### Example 4

### Testing of the Unspecific Toxicity of rhAFP/Ptx Complexes Against Normal CBA Mice Splenocytes In Vitro as Compared to Ptx Standalone

Normal CBA mice spleen cells were incubated for 48 h with various doses of rhAFP, Zn-rhAFP, Ptx or equimolar 1:1 rhAFP/Ptx or Zn-rhAFP/Ptx complexes. Viable cells were visualized by trypan blue staining. The data were expressed as percentage of viable cells in experimental cell cultures relatively to untreated control (FIG. 1B). These data distinctly demonstrated that Zn-rhAFP as well as rhAFP standalone induced dose-dependent stimulation of growth of the normal splenocytes reaching of 40% of control. Paclitaxel standalone demonstrated significant cytotoxic effect against normal splenocytes showing strong dose-dependent growth suppression (FIG. 1B). Treatment of spleen cells with equimolar combination of Zn-rhAFP/Ptx practically completely abrogated Ptx-induced cytotoxicity at low doses (less than 5.0 μM and significantly attenuated high-dose Ptx-induced cytotoxicity. Similar effects were observed for Zn-free rhAFP, rhAFPo and eAFP species. Taking into account ability of AFP to take up Ptx and that intact resting splenocytes do not express membrane AFP-receptors, we conclude that this effect is due to scavenger effect of rhAFP operating by elimination of free Ptx from the cell culture preventing thus its cell penetration and leading to abrogation of the unspecific toxicity.

### Example 5

### Tumor-Suppressive Activity of Combined rhAFP/Ptx and Zn-rhAFP/Ptx Preparations for Various Types of Tumor Cells

Tumor cell lines: human mammary carcinoma MCF-7, human hepatoma HepG2, and rat glioma C6—were originated from the American Type Culture Collection (ATCC). All cell lines were maintained in the complete DMEM or RPMI-1640 media supplemented with L-glutamine and 10% fetal calf serum (FCS) in humidified 5% CO₂ atmosphere. For a passage, adherent cells were incubated in the EDTA/0.25% trypsin solution, then washed and plated out for experiments. Prior to addition of reagents, cells were washed with the fresh media with 2% FCS, plated onto 96-well polystyrene plates (Costar, USA) at a density of 3×10³ cell/well in 180 μL of complete medium with 2% FCS and incubated for 2 h. Then 20 μL of rhAFP or Zn-rhAFP rhAFP or Zn-rhAFP solution in the phosphate buffered saline (PBS) with the appropriate protein concentration were added in the wells and incubated for 48 h. Cell proliferation was assessed by [³H]-thymidine (³H-TdR) incorporation assay. For the last 4-6 h of incubation, 1 μCi of ³H-TdR (Isotope, Russia) was added into the each well. Cells were harvested and the ³H-TdR incorporation was measured by using the liquid scintillation counter LKB 1209 Rackbeta (LKB, Sweden). Results were expressed as mean counts per minute (cpm)±standard error of the mean of triplicate cultures or as a percentage of ³H-TdR incorporation in the experimental cell cultures relatively to the untreated control, as described (Semenkova et al., Tumour Biol. 1997; 18: 261-74).

Cell viability was assessed by the Promega CellTiter 96 AQueous Non-Radioactive Cell Proliferation MTS assay (Promega, USA) according to manufacturer's instructions. Cells were incubated with various AFP samples for 48 h as described above, followed by incubation with MTS-test reagents and measuring of the light adsorption of the formasan, appeared in the lysed cell supernatants at 492 nm by using VICTOR-1420 multilabel counter (Wallac, Finland). Intact cell cultures incubated without additions were taken as a control. By such a way the quantity of viable cells capable to internalize MTS dye was determined. Results were expressed as the mean optical density (OD)±the standard error of the mean triplicate cultures. The extent of cell viability was calculated as an average OD in experimental wells after addition of testing compounds in relation to untreated control wells, that was taken as 100%

To evaluate the efficacy of preparations rhAFP/Ptx and Zn-rhAFP/Ptx we compared the cytotoxic effect of Ptx alone or that in combination with rhAFP or Zn-rhAFP for taxol-resistant human hepatoma HepG2 cells, human breast carcinoma cells MCF-7, and rat glioma C6 cell lines in vitro. RhAFP or Zn-rhAFP were incubated with various doses of Ptx at 37° C. for 1 h to allow complex formation with molecular ratios rhAFP:Ptx 1:1; 1:3 or 1:5 and thereafter the mixtures were introduced into the cell culture for 48 hrs. Cytotoxicity was measured by MTS assay.

### Human Mammary Carcinoma MCF-7 Cells

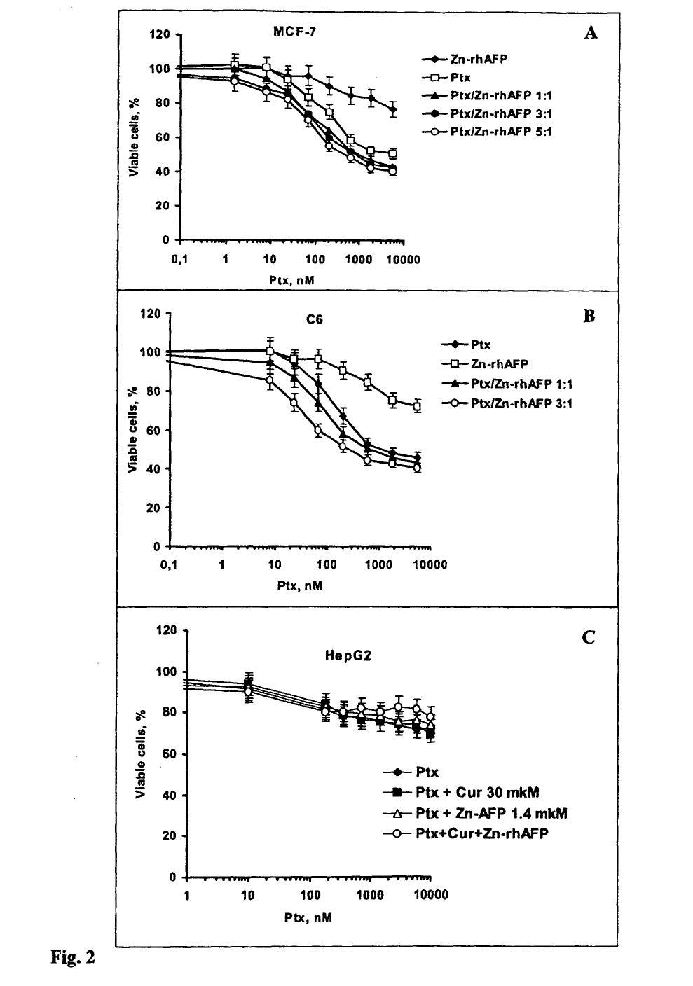

Human mammary carcinoma cell line MCF-7 revealed significant resistance to paclitaxel, showing lack of dose-dependence at high doses of Ptx (FIG. 2A). We tested the ability of rhAFP to overcome paclitaxel resistance. FIG. 2A shows the cytotoxic effect against MCF-7 cells of Ptx standalone or Ptx/Zn-rhAFP complexes prepared in molecular ratio 1:1; 1:3 or 1:5. Based on cytotoxic effects detected by MTS assay, the cytotoxic efficacy of Ptx/Zn-rhAFP complexes against MCF-7 cells as compared to Ptx standalone was additively enhanced in combined regimen, allowing significant decrease of the effective therapeutic dose. It should be noted that in Ptx/Zn-rhAFP (1:5) complex total concentration of Ptx was the same as in 1:1 and 1:3 complexes, but they differ on the content of Zn-rhAFP that is 5-fold and 3-fold lesser, correspondingly. Hence, Zn-rhAFP can take up more than one molecule of Ptx, reaching of at least five Ptx molecules for one Zn-rhAFP molecule without any decrease in total efficacy.

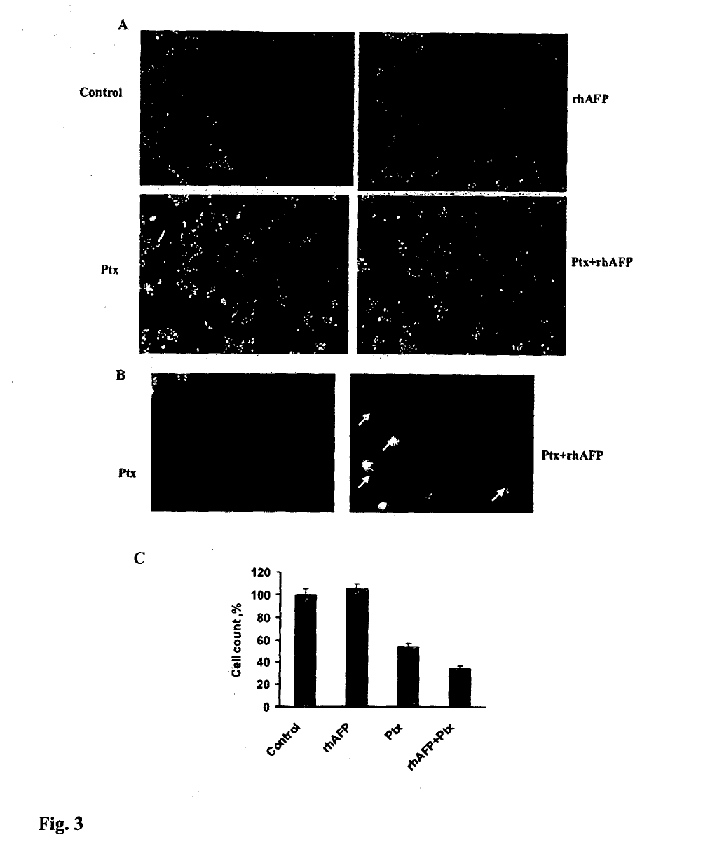

rhAFP/Ptx equimolar complex was also tested for its growth suppressive activity against tumor cells in vitro. FIG. 3 shows the photomicrograph of MCF-7 cells treated for 48 h with rhAFP, Ptx alone or with equimolar complex rhAFP/Ptx. FIG. 3A shows that combined rhAFP/Ptx treatment induces a notable increase in the total cell-killing effect showing decrease of the viable cell count reaching 35% of control in comparison with single component treatment with Ptx showing 54% of viable cells as compared to control or rhAFP standalone (FIG. 3C). FIG. 3B shows that combined treatment of MCF-7 cells with rhAFP/Ptx induces activation of apoptosis and sensibilization of the cells to the pro-apoptotic effects of Ptx. Apoptotic cells with condensed chromatin due to rhAFP/Ptx treatments are shown at FIG. 3B with arrows.

Rat Glioma C6

Rat glioma cell C6 showed significant increase in the total tumor suppression induced by combined Zn-rhAFP/Ptx versus Ptx standalone (FIG. 2B and Table 1). Combined Zn-rhAFP/Ptx treatment enhances more than twice tumor suppression activity against glioma C6 cells as compared with Ptx standalone.

HepG2 Human Hepatoma Cells

Human hepatoma HepG2 cells showed complete resistance to Ptx standalone as well as for combination of Zn-rhAFP/Ptx (FIG. 2C). To compare effect of the other well-known drug, curcumin (Cur), known to induce positive modulation of tumor-suppressive effects of Ptx we studied effect of complexes: Ptx/Cur; Ptx/Zn-rhAFP and Ptx/Zn-rhAFP/Cur on the growth of HepG2 cells. FIG. 2C shows that we failed to overcome chemoresistance of HepG2 cells to paclitaxel by such kind of combined therapy.

### Example 6

### Determination of the Anti-Tumor Efficacy of Treatment with Combined rhAFP/Ptx and Zn-rhAFP/Ptx Preparations for Various Types of Tumor Cells

Various doses of rhAFP/Ptx complexes were tested for their effectiveness for inhibition of human mammary carcinoma MCF-7, human hepatoma HepG2, and rat glioma C6 tumor cell growth in vitro. First, we optimized drug concentration and rhAFP amount to the extent that it would not generate an extensive cytotoxic effect alone. Then, the cells were seeded in 96-well plates at a density of 3×10³/well in 100 μl of medium for 24 hrs to allow adherence. The next day, rhAFP or Zn-rhAFP combined with Ptx at the molecular ratios of 1:1; 1:3 or 1:5 were added into the each well. After 48 h of incubation MTS assays were performed to determine survival rate of the cells. All the experiments were repeated at least three times. The results were expressed as mean±standard error (SE).

The coefficient of drug interaction (CDI) was calculated to analyze the effects of rhAFP and Ptx combinations. CDI was calculated by the equation: CDI=SR_{AFP/drug}×100%/SR_{AFP}×SR_{drug}, where SR_{AFP/drug} is the average survival rate of combination group, and SR_{AFP} or SR_{drug} are the average survival rates of the single agent groups. According to our evaluation, CDI values <1, =1 or >1 indicate that the combinations are synergistic, additive or antagonistic respectively. A CDI less than 0.7 indicate that the drugs are significantly synergistic.

The values of Zn-rhAFP/Ptx or rhAFP/Ptx complex concentration, which inhibited the cell viability by 50% (IC₅₀) when compared to untreated control, were calculated according to the Hill's equation (sigmoid model of concentration-response curve) from the curves of dependence of the relative percentage of viable cells from AFP concentration expressed as mean values from three independent experiments±SD.

To assess the efficacy of combined treatments with rhAFP/Ptx and Zn-rhAFP/Ptx complexes the coefficient of drug interaction (CDI) was calculated for all combinations, which was used to analyze the synergistically inhibitory effect of rhAFP/Ptx combinations for mammary carcinoma MCF-7 and C6 glioblastoma cells. We found that the most effective composition showed moderate synergy with CDI<1 (CDI˜0.8) for MCF-7 and CDI˜0.75 for C6 was Zn-rhAFP/Ptx*complex, whereas Ptx/rhAFP combination was resulted in additive effect with CDI˜1 reaching CDI of 0.9 for MCF-7 and CDI of 0.85 for C6.

The mean cytotoxic concentration (IC₅₀) corresponding to the half value of the total cytotoxic effect, that was calculated from three independent experiments, was determined for various compositions of rhAFP/Ptx and Zn-rhAFP/Ptx for breast cancer cell line MCF-7 and C6 glioma cells in vitro (Table 1). Both rhAFP and Zn-rhAFP standalone manifested cytostatic activity against MCF-7 cells reaching a value of 6.1 μM and 5.2 μM correspondingly, showing that Zn binding to rhAFP was resulted in the moderate enhance of the tumor-suppressive effect. Similarly, Zn-rhAFP/Ptx combinations were resulted in the more effective tumor suppression than rhAFP/Ptx compositions. Table 1 shows tumor growth-suppression effectiveness of various rhAFP/Ptx compositions for MCF-7 and C6 in vitro. The highest inhibitory activity was detected for MCF-7 cells for Zn-rhAFP/Ptx complexes with the IC₅₀ value of 1.5 μM whereas rhAFP/Ptx showed IC₅₀ of 2.2 μM, whereas Ptx standalone demonstrated a significantly lower activity with IC₅₀ of >10 μM showing distinct resistance of MCF-7 cells to paclitaxel (FIG. 2 and Table 1). C6 cells demonstrated more pronounced effects for Ptx standalone with IC₅₀ of 2.5 μM showing significant increase in effectiveness in complex reaching of IC₅₀ of 0.9 μM for rhAFP/Ptx (1:5) complex and IC₅₀ of 0.35 μM for Zn-rhAFP/Ptx (1:5) complex (FIG. 2 B and Table 1). Table 1 show summarized results for efficacy of Zn-rhAFP/Ptx combinations for various types of tumor cell lines in vitro.

Summarizing the results obtained for functional tests we conclude that both types of compositions under study Zn-rhAFP/Ptx and rhAFP/Ptx demonstrated the same type of biological activity resulting in growth suppression of breast cancer MCF-7 cells and glioma C6 cells in vitro, showing, however, less effective tumor suppressive activity for rhAFP-based compositions with Ptx as compared to Zn-rhAFP with Ptx.

TABLE 1
Coefficient for drug interaction (CDI) and IC₅₀ values
for compositions of paclitaxel with rhAFP or Zn-rhAFP
for mammary carcinoma cell line MCF-7.

| | rhAFP/Ptx | | | | Zn-rhAFP/Ptx ^{a)} | | | |
|---|---|---|---|---|---|---|---|---|
| rhAFP:Ptx | 0:1 | 1:1 | 1:3 | 1:5 | 0:1 | 1:1 | 1:3 | 1:5 |
| | MCF-7 | | | | | | | |
| CDI ^{b)} | — | 0.98 | 0.92 | 0.90 | — | 0.85 | 0.82 | 0.80 |
| IC₅₀ | >10 | 3.5 | 2.5 | 2.2 | >10 | 2.5 | 2.2 | 1.5 |
| (μM)^{c)} | | | | | | | | |
| Efficacy ^{d)} | — | 2.8 | 4.0 | 4.5 | — | 4.0 | 4.5 | 6.7 |
| | C6 | | | | | | | |
| CDI | — | 0.95 | 0.9 | 0.85 | — | 0.8 | 0.78 | 0.75 |
| IC₅₀ | 2.5 | 1.3 | 1.1 | 0.9 | 2.5 | 0.8 | 0.40 | 0.35 |
| (μM) | | | | | | | | |
| Efficacy | — | 2 | 2.3 | 2.8 | — | 3.2 | 6.3 | 7.1 |

^{a)} Molecular ratio of Zn:rhAFP was 4:1.
^{b)} Coefficient for drug interaction was calculated as described above.
^{c)}The values of rhAFP/Ptx concentration, which inhibited the cell viability by 50% when compared to unexposed control cells, were calculated from the sigmoidal curves of concentration dependence of the relative percentage of viable cells expressed as mean values from three independent experiments ± SD. SD does not exceed 10%.
^{d)} Efficacy coefficient was calculated as relative enhance of IC₅₀ in experimental group with combination of rhAFP/Ptx or Zn-rhAFP/Ptx in relation to Ptx standalone (given in column 0:1).

### Example 7

### Microscopic Evaluation of Effects of rhAFP-Based Paclitaxel Formulations on the Viability and Apoptosis of Breast Cancer Cells MCF-7

MCF-7 cells were seeded onto the microscopic plates Lab-Tek™ Chamber Slides (Thermo Fisher Scientific-Nunc, Germany) for 48 h to reach of 80% confluence and thereafter were subjected to 48-h treatments with Ptx dissolved in DMSO (10 nM), Zn-rhAFP (1.4 μM), or their combination Zn-rhAFP/Ptx (1.4 μM/10 nM). Similarly, the tests had been performed with non-glycosylated rhAFPo species. Thereafter the cells were fixed with 100% ice-cold acetone and stained with DAPI (Molecular probes, USA). Control cells were incubated without additions. Cells were visualized with fluorescent light microscope Axioplan (Zeiss, Germany) at ×100 or ×400 magnification. The quantity of the cells in the various experimental wells in the selected fields of view was assessed by using ImageJ software. FIG. 3 A, C shows that combined treatment of MCF-7 cells with Zn-rhAFP/Ptx was resulted in the significant enhance of killing effect reaching of 70% of cell death in Zn-rhAFP/Ptx treatment regimen whereas Ptx standalone induced 55% of cell death. FIG. 3B demonstrates that combined treatment of MCF-7 cells with Zn-rhAFP-Ptx was resulted in the significant enhance of the population of cells with apoptotic morphology. The arrows show apoptotic nuclei with fragmented chromatin characteristic of apoptosis (FIG. 3B).

### Conclusions:

Combined administration of Zn-rhAFP/Ptx results in significant enhance of cells with apoptotic morphology in population of breast carcinoma cells MCF-7 as compared to effect of Ptx alone.
AFP-receptor targeted delivery of Zn-rhAFP/Ptx amplifies cytotoxicity toward cancer cells and significantly decreases unspecific side toxicity.
We have proved that this formulation is more effective than pure paclitaxel to treat cancer. Zn-rhAFP/Ptx complex is soluble in water solutions and might be used for preparation of injectable forms.
Zn-rhAFP/Ptx complex is usable for targeting treatment of cancer of various location including neurological diseases because AFP can penetrate via blood brain barrier.

### Example 7

### Combined Cytotoxicity of Zn-Functionalized rhAFP with Various Chemotherapeutic Agents

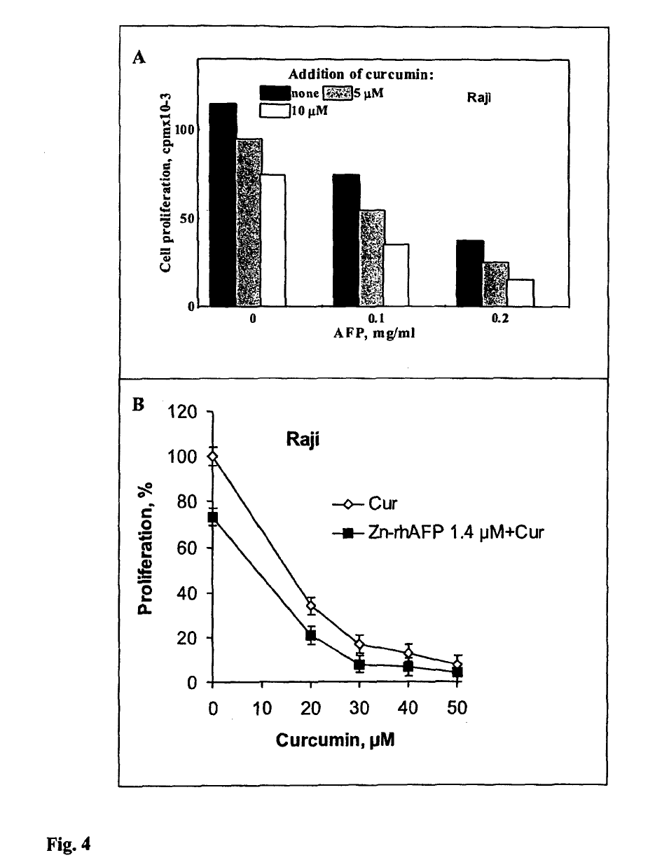

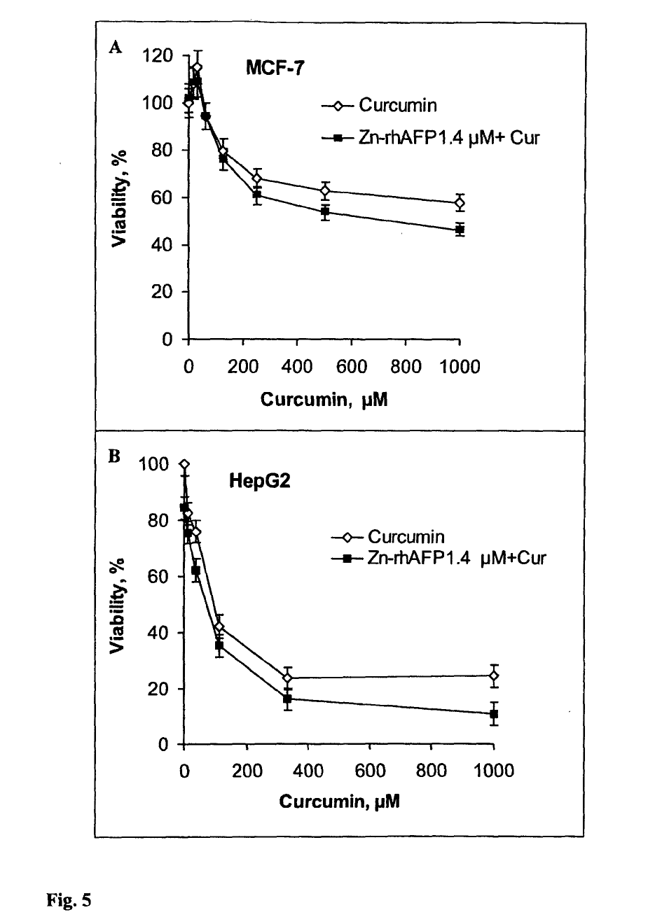

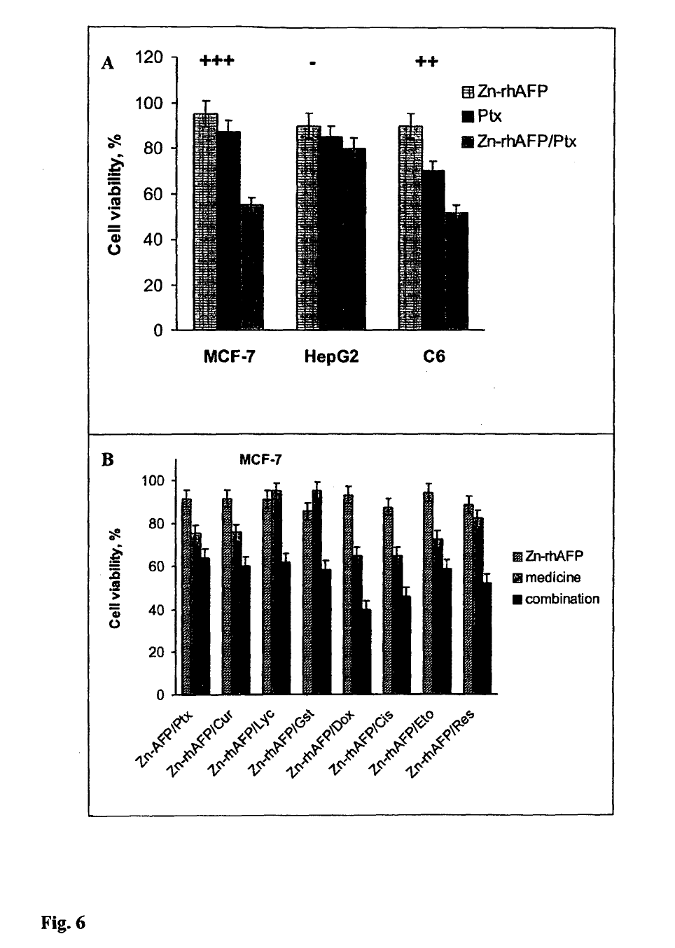

In order to confirm the hypothesis that the combination of Zn-rhAFP and various low water-soluble chemotherapeutic agents would reveal increased anti-tumor efficacy, different cytotoxic compounds were tested in combination with Zn-rhAFP for their cell-killing effect in MCF-7 cells in vitro. Different plant-derived chemotherapeutic agents: curcumin (Cur); genistein (Gst); lycopene (Lyc); paclitaxel (Ptx); and chemotherapeutic drugs: doxorubicin (Dox); cisplatin (Cis); etoposide (Eto), —were tested in combination with Zn-rhAFP (4:1) for their killing effects on various types of tumor cells in vitro. First, we optimized drug concentration and Zn-rhAFP amount to the extent that it would not generate an extensive cytotoxic effect alone. Then, MCF-7 cells were seeded in 96-well plates at a density of 3×10³/well in 100 μl medium for 24 hrs to allow adherence. The next day, Zn-rhAFP combined with different chemotherapeutic agents was added into the each well to obtain dose-dependence of cell survival rate. After 48 hrs of incubation MTS assays were performed to determine survival rate of the cells. Each combination of Zn-rhAFP/medicine was evaluated for anti-tumor efficacy by calculating coefficient for drug interaction (CDI) that was obtained from the dose-dependence curves of cell viability as described above (Example 6). Examples of dose-dependent growth-suppressive effects induced by combined treatment with Zn/rhAFP-curcumin of various types of tumor cells (Raji, FIG. 4; MCF-7, FIG. 5A and HepG2, FIG. 5B) are shown in FIG. 4 and FIG. 5. These data distinctly indicated that combined administration of Zn-rhAFP/curcumin was resulted in significant enhance of anti-tumor efficacy. FIG. 6A demonstrates the synergistic effects of combined treatments with Zn-rhAFP/Ptx for various types of human tumor cells (MCF-7, HepG2, C6). FIG. 6B shows synergistic tumor suppressive effects induced by combination of Zn-rhAFP with various anti-tumor drugs: paclitaxel, curcumin, lycopene, genistein, doxorubicin, cisplatin, etoposide against mammary carcinoma cells MCF-7. Distinct synergy was observed for certain combinations as assessed by calculated CDI values (Table 2).

TABLE 2
Coefficient for drug interaction (CDI) for combined effects of rhAFP
with various chemotherapeutics for mammary carcinoma cell line MCF-7
MCF7 + Zn-rhAFP 1.4 μM + drugs:

| medicine | Paclitaxel | Curcumin | Doxorubicin | Cisplatin | Etoposide | Genistein | Lycopene | Resveratrol |
|---|---|---|---|---|---|---|---|---|
| CDI | 0.80 | 0.88 | 0.66 | 0.93 | 0.86 | 0.82 | 0.71 | 0.72 |

Table 2 shows that combination of Zn-rhAFP together with various anti-tumor drugs were resulted in significant enhance of efficacy of treatment. According to the value of the coefficient of drug interaction (CDI), which was used to analyze the synergistically inhibitory effect of drug combinations, we found that the combination of Zn-rhAFP with doxorubicin, resveratrol and lycopene showed significant synergy (CDI<0.7), paclitaxel, etoposide, cisplatin, curcumin and genistein demonstrated not so pronounced synergistic effect with rhAFP (CDI<1).

### Conclusions:

Combined administration of low-water soluble chemotherapeutic drugs with Zn-functionalized rhAFP produces significant enhance in the anti-tumor efficacy against resistant human breast cancer cells.
Combination of various water insoluble plant-derived medicines with Zn-rhAFP markedly increases water solubility, bioavailability and cytotoxicity of the drug against resistant human breast cancer cell lines.

### Example 8

### Immunofluorescence Determination of Cytoplasmic and Nuclear Translocation of Activated NF-μB Due to Zn-rhAFP and Zn-rhAFP/Ptx Treatments

MCF-7 cells were seeded on the microscopic plates Lab-Tek™ Chamber Slides (Thermo Fisher Scientific-Nunc, Germany) and cultivated at 37° C. at 5% CO₂ atmosphere for 48 h to allow cell adaptation to reach 80% confluence. To test effects of chemotherapeutic preparations on the activation and translocation of NF-κB, cells were treated for 2 h with Ptx (10 nM), curcumin (30 μM); Zn-rhAFP (1.4 μM), or their combination Zn-rhAFP/Ptx/Cur, after which they were fixed with 100% ice-cold acetone. Control cells were incubated without additions and their NF-κB content was taken as 100%. Activated NF-κB was detected by treatment of the cells with phospho NF-κB p65 rabbit polyclonal antibody (Cell signaling technologies, USA) followed by the secondary goat anti-rabbit antibodies Ab-Alexa594 (Molecular Probes, USA). Nuclei were visualized by staining with DAPI (Molecular probes, USA). Cells were visualized at magnification ×400 by fluorescent microscope Axioplan (Zeiss, Germany) equipped with corresponding color filter kits. Distribution throughout the cell of the activated p65 was assessed by the ImageJ software and was taken as 100% in the control samples (cytosol+nucleus).

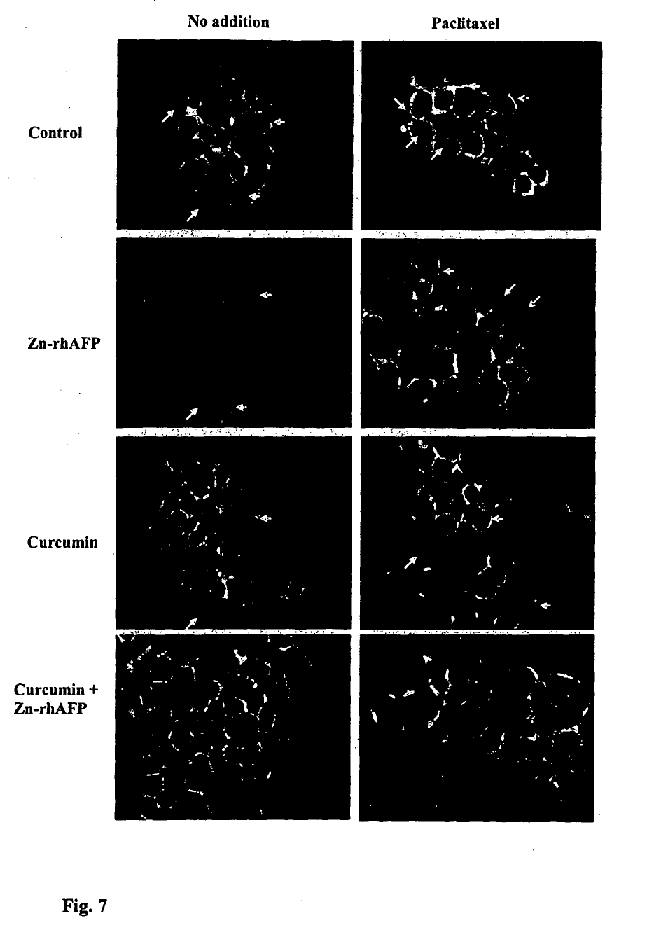

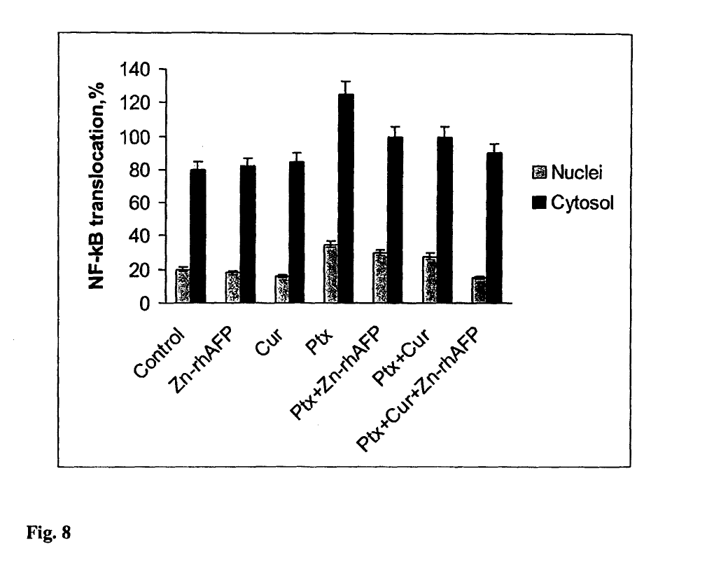

The microphotography of stained with anti-p65 MCF-7 cells after corresponding treatments with Zn-rhAFP/Ptx/Cur (FIG. 7) combinations shows that Ptx induced significant enhance of activated p65 localized in the nuclear region. Treatment with Zn-rhAFP or/and curcumin was resulted in significant decrease of activated RelA in the cell nucleus with simultaneous its cytoplasmic translocation, showing its deactivation. The most effective inhibition of Ptx-induced nuclear translocation of p65 was observed upon combined administration of the both compounds Zn-rhAFP and curcumin, showing 60% decrease of activated p65 in the nucleus of the MCF-7 cell (FIG. 8). Therefore paclitaxel activated NF-κB in breast cancer cells, and rhAFP/curcumin inhibited it by inducing its release from the nucleus of activated cells. The molecular mechanisms of this effect are under study, but the experimental data obtained for various types of tumor cells induced activation of NF-κB by different stimuli demonstrated similar suppressive effects induced by rhAFP treatment. These data indicate that Zn-rhAFP inhibits activation of p65 and its translocation in the nucleus sensitizing tumor cells to cytotoxic effects of drugs. It is known that activation of NF-κB induced by various cytotoxic drugs enhances significantly tumor cell resistance to treatments. Factors involved in inhibition of NF-κB activation can sensitize tumor cells to apoptotic stimuli. Our data show that Zn-rhAFP can sensitize tumor cells to chemotherapeutic drugs by blocking drug-induced NF-κB activation, which is involved in tumor cell chemoresistance.

### Conclusions:

Our data indicated that Zn-rhAFP sensitizes tumor cells to chemotherapeutic drugs operating by inhibition of constitutive or drug-induced NF-κB activation.
Zn-rhAFP and curcumin operates synergistically to inhibit of constitutive or drug-induced NF-κB activation.
Zn-rhAFP sensitizes tumor cells to cytotoxicity induced by chemotherapeutic drugs by blocking NF-κB activation.

### Example 9

### Combined Administration of Curcumin with Zn-rhAFP Enhances Significantly Endocytosis of Curcumin in the Nuclei of Tumor Cells

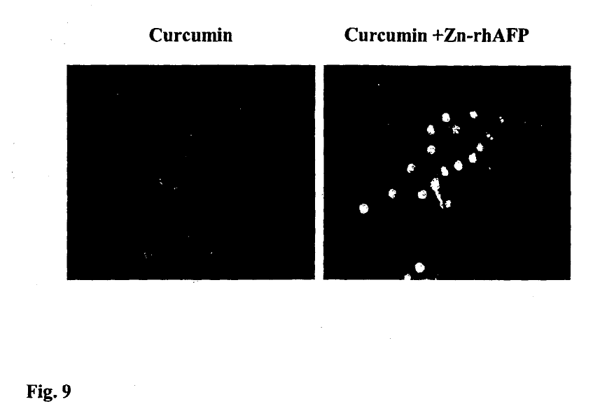

Endocytosis of curcumin and Zn-rhAFP/curcumin complexes by MCF-7 cells was studied by fluorescence microscopy by monitoring of own green fluorescence of curcumin detected by using of corresponding green filter system in fluorescent Axioplan microscope, Zeiss (Germany). FIG. 9 demonstrates that combined administration of Zn-rhAFP with low doses of curcumin induced the notable enhance of endocytosis of curcumin in the nuclei of MCF-7 cells as compared with the same dose of curcumin introduced without Zn-rhAFP. These data demonstrate that formation of rhAFP-complexes facilitates penetration of curcumin inside the cell and directly in the cell nucleus, showing targeting carrier function of rhAFP.

### Example 10

### Determination of Thermodynamic Parameters of Heat Denaturation of Non-Covalent Complexes of rhAFP and Small Hydrophobic Molecules. Effect of Formation of Non-Covalent Complexes of rhAFP with Zn(II), Paclitaxel, and Curcumin Binding on the Protein Tertiary Structure

Calorimetric measurements were performed using a DASM-4 differential capillary scanning calorimeter equipped with cells of 0.464 ml working volume (Pushchino, Russia). Calorimetric runs of the samples were carried out within a temperature range of 1 to 100° C. at a heating rate of 1.0 K/min. Calculations of the specific excess heat capacity function C_{p.exe}(T), specific denaturation heat Q_{d}, and the theoretical deconvolution analysis of the determined function C_{p.exe}(T) were performed as described earlier in (Dudich et al., Biochemistry 1999; 38: 10406-10414).

It is very difficult to track colorless ligand molecules for their binding with large carrier molecules. Usually, to manage ligand tracking it is necessary to produce staining of ligand with any fluorescent or radioactive label in order to detect their binding to the carrier macromolecule. We have developed a technique of controlling complex formation of AFP and colorless hydrophobic compounds by monitoring a process of conformation changes in tertiary structure of the macromolecule upon the process of heat denaturation. Specific conformational state of the ligand-free rhAFP molecule could be approximated by parameters of the “molten globule” conformation that is characterized by the tertiary structure unstable to the temperature melting denaturation injury. AFP can bind metals and small hydrophobic molecules inducing significant stabilization of the tertiary structure of the protein in respect to the heat melting. These conformational changes can be monitored by measuring of the heat melting parameters, such as enthalpy of denaturation transition and denaturation transition temperature, which are characteristic parameters for the conformational state of a protein macromolecule. Several metal ions were studied in this way. Detailed thermodynamic data for Zn ions illustrate the typical behavior of different metal complexes.

FIG. 10A shows calorimetric scans of intact ligand-free rhAFP, Zn-AFP/Zn and Zn-AFP/Ptx complexes. It is seen, that ligand removal drastically changed the melting pattern of rhAFP. The addition of Ptx to rhAFP practically completely recovered initial shape of the protein melting pattern, characteristic of the intact rhAFP molecule (FIG. 10A). The similar results were obtained for Zn-rhAFP/curcumin (FIG. 10B). Non-covalent Zn-rhAFP/Cur and Zn-rhAFP/Ptx complex formation leads to stabilization of tertiary structure of the rhAFP molecule.

Thermodynamic parameters obtained for various rhAFP samples under these conditions are presented in Table 3. It is seen that ligand removal led to the significant decrease in values of denaturation enthalpies and transition temperatures of both transitions. This data indicate that changes in thermodynamic parameters of rhAFP, induced by the ligand removal, reflect destabilization of the rhAFP tertiary structure. These conformational changes were reversible—the ligand addition practically completely recovered the initial shape of the melting pattern characteristic of the intact rhAFP molecule. It is seen that Zn binding only partially affects recovery of the native structural stability characteristic of the intact rhAFP molecule (FIG. 9A, B).

TABLE 3
Effect of Zn ions and small hydrophobic ligands interaction
on the rhAFP heat melting thermodynamic parameters.

| Sample* | ΔH (kJ · mol⁻¹) | Tₘₐₓ ° C. | ΔT_{1/2} ° C.** |
|---|---|---|---|
| rhAFP | 694 | 71.9 | 7.0 |
| rhAFP + Zn | 716 | 74.3 | 5.9 |
| rhAFP + Zn + Ptx | 738 | 77.9 | 3.4 |
| rhAFP + Zn + Cur | 776 | 76.5 | 6.2 |
| eAFP | 790 | 80.2 | 5.8 |

*Protein concentration was 2.0 mg/ml in phosphate buffered saline, pH 7.4. rhAFP was defatted by HCl/charcoal treatment before measurements.
Errors in enthalpies are approximately ±6% and in denaturation temperatures ±0.5° C.
**Half width of the denaturation transition peak
Relative amount of ligands: Zn/rhAFP = 4/1; Ptx/rhAFP = 1/1; Cur/rhAFP = 1:1.

On the other hand, loading of rhAFP/Zn with Ptx or Cur led to the notable increase in the values of temperatures and enthalpies of the distinct transitions, showing certain stabilization of the rhAFP structure respectively to the heat melting. These data also show that Zn and Ptx or curcumin utilize different active sites on the surface of the AFP molecule for their binding. Tertiary structure of the rhAFP undergoes reversible conformational changes induced by ligand removal leading to destabilization of the protein conformation. Addition of hydrophobic ligands, such as paclitaxel or curcumin, completely recovered the initial tertiary structure parameters characteristic for the intact natural eAFP (FIG. 10, Table 3). These data show that rhAFP forms unique non-covalent complexes with Zn and Ptx and/or curcumin leading to formation of the more stable tertiary structure resistant to heat denaturation. Biological activity data show that rhAFP structurized with ligands is significantly more active than that which was not stabilized with metal ions and small hydrophobic ligands (Examples 5-7).

### Conclusions:

Binding of Zn(II) ions to the rhAFP molecule induces notable conformational change of the tertiary structure of the rhAFP showing complex formation and demonstrating structural difference between Zn-rhAFP complex and intact rhAFP molecule.
Interaction of paclitaxel or curcumin with rhAFP or Zn-stabilized rhAFP is accompanied with significant conformational change in the tertiary structure of the rhAFP molecule showing non-covalent complex formation resulting in significant enhance of its conformational stability.
Paclitaxel or curcumin binds to rhAFP and Zn-rhAFP by forming high affinity non-covalent complex via hydrophobic interaction allowing drug solubilization and its targeting delivery directly to cancer cells.
Paclitaxel-loaded Zn-rhAFP markedly increases water solubility, bioavailability and cytotoxicity of the drug exemplified targeting tumor suppressive affects against breast cancer and glioblastoma tumor cells in vitro.
Due to selective targeting delivery of noncovalent complexes of Zn-rhAFP/Ptx to the AFP-receptor-expressing cells, in particular tumor cells, these non-covalent complexes have a significantly lower non-specific toxicity to normal cells.
Zn-rhAFP/Ptx (rhAFP/Ptx) complex is usable for targeting treatment of cancer of various location including neurological tumors because AFP can penetrate via blood brain barrier.
Paclitaxel and curcumin bind human recombinant alpha-fetoprotein (rhAFP) by forming high affinity non-covalent complex via hydrophobic interaction allowing drug solubilization and its targeting delivery directly to cancer cells avoiding normal cells.
Paclitaxel-loaded rhAFP markedly increases water solubility, bioavailability and cytotoxicity of the drug against resistant human breast cancer cell lines.
The possibility of metal ion-stabilized AFP to bind small hydrophobic molecules, such as paclitaxel could be utilized for design of the novel forms of targeting drug-delivery systems allowing tracking of water-insoluble cytotoxic compounds selectively in tumor cells avoiding normal cells.

### REFERENCES

Benevolensky S. V., Marchenko A. N., Kozlov D. G., Zatsepin S. S., Shingarova L. N., Dudich I. V., Semenkova L. N., Dudich D. I., Tatulov E. B., Dudich E. I. Recombinant alpha-fetoprotein and method of preparing U.S. Pat. No. 7,910,327, 2011.
Bennett J. A., Semeniuk D. J., Jacobson H. I., Murgita R. A. (1997) Similarity between natural and recombinant human alpha-fetoprotein as inhibitors of estrogen-dependent breast cancer growth, Breast Cancer Res. Treat., 45: 169-179.
Constab Pharmaceuticals Inc. WO2007056852 A1.
Ceramoptec Industries Ltd. WO2010129337 A2.
Desai N. P., Soon-Shiong P., Trieu V. Compositions and methods of delivery of pharmacological agents U.S. Pat. No. 7,923,536, 2011.
Desai N. P., Soon-Shiong P. D., Tapas K. Methods and compositions for treating proliferative diseases U.S. Pat. No. 7,780,984, 2010.
Desai N. P., Soon-Shiong P., Trieu V. Compositions and methods of delivery of pharmacological agents U.S. Pat. No. 7,820,788, 2010.
Desai N. P., Soon-Shiong P. Combinations and modes of administration of therapeutic agents and combination therapy U.S. Pat. No. 7,758,891, 2010.
Deutsch H. F. (1991) Chemistry and biology of α-fetoprotein. Adv. Cancer. Res. 56: 253-311.
Dudich E., Semenkova L., Dudich 0.1., Tatulov E. Recombinant alpha-fetoprotein and compositions thereof. US Patent Application 20110159112 A1, 2011.
Dudich E., Dudich I., Semenkova L., Benevolensky S., Morozkina E., Marchenko A., Zatcepin S., Dudich D., Soboleva G., Khromikh L., Roslovtceva O., Tatulov E. (2012) Engineering of the Saccharomyces cerevisiae yeast strain with multiple chromosome-integrated genes of human alpha-fetoprotein and its high-yield secretory production, purification, structural and functional characterization. Prot. Exp. Purif., 84: 94-107.
Dudich I. V., Tokhtamysheva N., Semenkova L. N., Dudich E. I., Hellman J., Korpela T. (1999) Isolation and structural and functional characterization of two stable peptic fragments of human alpha-fetoprotein, Biochemistry 38:10406-10414.
Dudich E. I., Semenkova L. N., Dudich I. V., Denesyuk A. I., Tatulov E. B., Korpela T. (2006) Alpha-fetoprotein antagonizes X-linked inhibitor of apoptosis protein anticaspase activity and disrupts XIAP-caspase interaction, FEBS J. 273: 3837-3849.
E. I. Dudich, L. N. Semenkova, I. V. Dudich, E. A. Gorbatova, N. A. Tokhtamysheva, E. B. Tatulov, M. A. Nikolaeva, G. T. Sukhikh, (1999) α-Fetoprotein causes apoptosis in tumor cells via a pathway independent of CD95, TNFR1 and TNFR2 through activation of caspase-3-like proteases, Eur. J. Biochem. 266:750-761.
Feldman N. B., Kiselev S. M., Gukasova N. V., Posypanova G. A., Lutsenko S. V., Severin S. E. (2000) Antitumor activity of alpha-fetoprotein conjugate with doxorubicin in vitro and in vivo. Biochemistry (Russia); 65(8):967-71.
Godovannyi A. V., Vorontsov E. A., Gukasova N. V., Pozdnyakova N. V., Vasilenko E. A., Yabbarov N. G., Severin S. E., Severin E. S., and Gnuchev N. V. (2012) Antitumor Activity of Targeted Nanosome Delivery Systems Based on Poly(lactic-co-glycolic Acid) Nanoparticles, Paclitaxel, and a Recombinant Alpha-Fetoprotein Fragment. Nanotechnologies in Russia, 7: 76-84.
Kratz F., Elsadek B. Clinical impact of serum proteins on drug delivery. J. Control. Release 2012; 157: 4-28.
Kratz F. Albumin as a drug carrier: Design of prodrugs, drug conjugates and nanoparticles. J. Control. Release 2008; 132: 171-183.
Metzler, D., Biochemistry, p. 311, 2001, Academic Press, New York.
Mizejewsky G. J. (2002) Biological role of α-fetoprotein in cancer: prospects for anticancer therapy. Expert Rev. Anticancer. Ther. 2 89-115.
Mizejewski G. J. Biological role of alpha-fetoprotein in cancer: Prospects for anticancer therapy. Expert Rev. Anticancer Ther. 2: 709-735.
Mizejewski G. J. (2001) Alpha-fetoprotein Structure and Function: Relevance to Isoforms, Epitopes, and Conformational Variants Exp Biol Med Vol. 226(5):377-408,
Permyakov S. E., Oberg K. A., Cherskaya A. M., Shavlovsky M. M., Permyakov E. A., Uversky V. N. (2002) Human alpha-fetoprotein as a Zn(2+)-binding protein. Tight cation binding is not accompanied by global changes in protein structure and stability. Biochim Biophys Acta 1586: 1-10.
Semenkova L. N, Dudich E. I., Dudich I. V., Tokhtamysheva N. A., Tatulov E. B., Okruzhnov Y. A., Garcia-Foncillas J., Palop-Cubillo J.-A., Korpela T. (2003) Alpha-fetoprotein positively regulates cytochrome c-mediated caspase activation and apoptosome complex formation, Eur. J. Biochem. 70: 4388-4399.
Uversky V. N., Narizhneva N. V., Ivanova T. V., Tomashevski A. Yu. (1997) Rigidity of human AFP tertiary structure is under ligand control. Biochemistry 36: 13638-13645.

1. A pharmaceutical formulation comprising a metal-ion stabilized recombinant alpha-fetoprotein (AFP), wherein one or more transition metal-ions are non-covalently bound to the AFP, and wherein the AFP additionally carries non-covalently bound effectors or drug molecules.

2. The pharmaceutical composition according to claim 1, wherein the AFP is a recombinant human AFP (rhAFP), produced by recombinant technology in eukaryotic or prokaryotic cells.

3. The pharmaceutical composition according to claim 1, wherein the stabilizing metal ion is selected from the group consisting of Ni, Zn, Cd, Cu, Mn, Co, Fe, Pt, and a mixture of anyone of them.

4. The pharmaceutical composition according to claim 1, wherein the stabilizing metal ions or their mixture are present in molar ratio of 1 to 20 metal ions per one AFP-molecule.

5. A water-soluble pharmaceutical composition comprising a molecular assembly of metal ion-stabilized AFP and effector and/or drug molecules according to claim 1, wherein the composition has AFP's natural character to a selective targeting to the cells expressing AFP-receptors.

6. The water-soluble pharmaceutical composition according to claim 5, wherein the effector or drug molecule is paclitaxel or a derivative thereof, and wherein the molar ratio of AFP to paclitaxel or a derivative thereof, is from 1:1 to 1:20.

7. The water-soluble pharmaceutical composition according to claim 5, wherein the effector or drug is curcumin, resveratrol, genistein, lycopene, doxorubicin, etoposide, cisplatin in molar ratio of AFP to ligand from 1:1 to 1:20.

8. The pharmaceutical composition according to claim 5, wherein the composition additionally contains a surplus of formulating agents selected from the group consisting of non-metal-stabilized AFP, albumin, and another common formulating agent.

9. The pharmaceutical composition according to claim 5, wherein the molecular assembly contains two or more different drugs molecules, and wherein the molecular assembly has a synergistic effect with different drugs on the target cells.

10. The method of preparing pharmaceutical composition containing a metal stabilized human recombinant AFP (rhAFP) comprising the steps of:
(i) preparation of deionized and defatted rhAFP;
(ii) incubation of deionized and defatted rhAFP (i) in a zinc-ion solution;
(iii) addition of one or more kinds of toxins, drugs or effectors in an appropriate solution to the zinc ion loaded rhAFP;
(iv) removal of non-bound ligands from the complex; and
(v) preparation of the final pharmaceutical composition.
